(19) United States
(12) Patent Application Publication
(10) Pub. No.: US 2020/0033360 A1
(43) Pub. Date: Jan. 30, 2020

(54) COMPREHENSIVE AND QUANTITATIVE LIPID AND TOCOPHEROL ANALYSIS

(71) Applicant: Metabolon, Inc., Morrisville, NC (US)

(72) Inventors: Elizaveta FREINKMAN, Cary, NC (US); Anne M. EVANS, Cary, NC (US); Kelli GOODMAN, Raleigh, NC (US); Richard J. ROBINSON, Morrisville, NC (US)

(21) Appl. No.: 16/491,471

(22) PCT Filed: Mar. 28, 2018
(86) PCT No.: PCT/US18/24767

§ 371 Date: Sep. 5, 2019

Related U.S. Application Data
(60) Provisional application No. 62/592,639, filed on Nov. 30, 2017.
(60) Provisional application No. 62/558,415, filed on Sep. 14, 2017.
(60) Provisional application No. 62/512,776, filed on May 31, 2017.
(60) Provisional application No. 62/479,534, filed on Mar. 31, 2017.

(51) Int. Cl. G01N 33/68; G01N 33/92

(52) U.S. Cl. CPC G01N 33/6848; G01N 33/92

(57) ABSTRACT

A method for determining in a sample, by mass spectrometry, the presence, absence or amount of one or more lipid species from one or more lipid classes is described herein. The one or more lipid classes are selected from the group consisting of wax esters (WE), squalene (SQ), triacylglycerols (TAG), diacylglycerols (DAG), free fatty acids (FFA), cholesteryl esters (CE), cholesterol (CH), and combinations thereof. The method includes a) subjecting the sample to an ionization source to produce one or more ions detectable by mass spectrometry from each of the one or more lipid species; b) measuring, by mass spectrometry, the amount of the one or more ions from each of the one or more lipid species; and c) using the measured amount of the one or more ions to determine the presence, absence or amount of each of the one or more lipid species.

### CROSS-REFERENCE TO RELATED APPLICATIONS

This application claims the benefit of U.S. Provisional Patent Application No. 62/479,534, filed Mar. 31, 2017, U.S. Provisional Patent Application No. 62/512,776, filed May 31, 2017, U.S. Provisional Patent Application No. 62/558,415, filed Sep. 14, 2017, and U.S. Provisional Patent Application No. 62/592,639, filed Nov. 30, 2017, the entire contents of each of which are hereby incorporated herein by reference.

### BACKGROUND

The following information to describe the background of the invention is provided to assist the understanding of the invention and is not admitted to constitute or describe prior art to the invention.

Sebum is a lipid-rich secretion produced by sebocytes, specialized cells of sebaceous glands. In humans, sebum coats the surface of skin and contributes to antimicrobial defense, water retention, photoprotection, and wound healing. The sebum lipidome is comprised of a complex mixture of lipids. The sebum lipid composition differs among species, likely as a result of the various functional requirements. Human sebum lipids include not only free fatty acids (FFAs), cholesterol (CH), cholesteryl/cholesterol esters (CEs), and di- and triacylglycerols (DAGs and TAGs) but also wax esters (WEs) and squalene (SQ), which are unique to the sebum. In addition to these lipids, sebum contains lipid-soluble molecules, such as tocopherols. Sebum characterization has involved thin-layer chromatography to separate the lipid classes, followed by determination of bulk fatty-acid composition within each class without identifying individual lipid species (Stewart et al., J Invest Dermatol. 1986; 87(6):733-6). More recently, methods combining liquid chromatography (LC) and mass spectrometry (MS) have been used to characterize sebum lipids (Camera et al, J Lipid Res. 2010; 51(11); 3377-88), but these methods report only the sum composition and do not identify the fatty acid composition of lipid species. Further, these methods provide only relative quantitation, and the fatty acid composition of the sebum lipidome cannot be quantified across lipid classes. In addition, the methods require a prior separation or purification step. Here, we present methods that simultaneously identify the fatty acid composition of the major lipid species in sebum and measure the absolute concentration of these molecules and the lipid-soluble tocopherols.

Described herein are methods and assays for the detection and quantitation of lipids and tocopherols in a sebum sample.

### SUMMARY

In a first aspect of the invention, a method for determining in a sample, by mass spectrometry, the presence, absence or amount of one or more lipid species from one or more lipid classes selected from the group consisting of wax esters (WE), squalene (SQ), triacylglycerols (TAG), diacylglycerols (DAG), free fatty acids (FFA), cholesteryl esters (CE), cholesterol (CH), and combinations thereof, comprises multiple steps. The steps include subjecting the sample to an ionization source under conditions suitable to produce one or more ions detectable by mass spectrometry from each of the one or more of the lipid species; measuring, by mass spectrometry, the amount of the one or more ions from each of the one or more lipid species; and using the measured amount of the one or more ions to determine the presence, absence or amount of each of the one or more lipid species in the sample.

In a second aspect of the invention, a method for determining in a sample, by mass spectrometry, the presence, absence or amount of one or more lipid species and one or more tocopherols comprises multiple steps. The one or more lipid species are from one or more lipid classes, which are selected from the group consisting wax esters (WE), squalene (SQ), triacylglycerols (TAG), diacylglycerols (DAG), free fatty acids (FFA), cholesteryl esters (CE), cholesterol (CH), tocopherols (TOC), and combinations thereof. The one or more tocopherols are selected from the group consisting of alpha-tocopherol, beta-tocopherol, gamma tocopherol, and delta-tocopherol, and combinations thereof. The steps include subjecting the sample to an ionization source under conditions suitable to produce one or more ions detectable by mass spectrometry from each of the one or more of the lipid species and one or more tocopherols; measuring, by mass spectrometry, the amount of the one or more ions from each of the one or more lipid species and one or more tocopherols; and using the measured amount of the one or more ions to determine the presence, absence or amount of each of the one or more lipid species and one or more tocopherols in the sample.

In a feature of the first and second aspect, the one or more lipid classes comprise wax esters (WE), triacylglycerols (TAG), diacylglycerols (DAG), cholesteryl esters (CE), or combinations thereof, and the method further comprises determining the number of carbons and double bonds of one or more fatty acids of the one or more lipid species.

In another feature, the sample is a sebum sample or a sebocyte sample. In yet another feature, the presence, absence, or amount of the one or more lipid species from the one or more lipid classes is determined from a single injection. In a further feature, the one or more lipid species from one or more lipid classes comprise wax esters, and the method further comprises determining the fatty alcohol composition of the wax ester. In still a further feature, the one or more lipid species from the one or more lipid classes comprise diacylglycerols, and the method further comprises determining the number of carbons and double bonds for two fatty acids of the diacylglycerol.

In an additional feature, the amount of one or more lipid species from at least two lipid classes is determined. With respect to this feature, the at least two lipid classes comprise squalene and wax esters. With further regard to this feature, the at least two lipid classes comprise squalene and diacylglycerols. Still further, with regard to this feature, the at least two lipid classes comprise squalene and triacylglycerols, squalene and free fatty acids, squalene and cholesteryl esters, squalene and cholesterol, wax esters and triacylglycerols, wax esters and diacylglycerols, wax esters and free fatty acids, wax esters and cholesteryl esters, or wax esters and cholesterol.

In further features, the amount of one or more lipid species from three or more, four or more, five or more, or six or more lipid classes is determined. In another feature, the amount of one or more lipid species from each of the lipid classes CH, CE, WE, SQ, TAG, DAG, and FFA are determined. In yet another feature, the amount of one or more lipid species selected from the group consisting of CH, CE, WE, SQ, TAG, DAG, and FFA are determined, and the amount of one or more TOC species selected from the group consisting of alpha-tocopherol, beta-tocopherol, gamma tocopherol, and delta-tocopherol are determined. In a still further feature, the amount of twenty or more lipid species is determined.

In another feature, one or more internal standards are used to determine the amount of the one or more lipid species in the sample. With regard to this feature, the one or more internal standards are selected from one or more lipid classes selected from the group consisting of wax esters (WE), squalene (SQ), triacylglycrides (TAG), diacylglycerols (DAG), free fatty acids (FFA), cholesteryl esters (CE) and combinations thereof. With further regard to this feature, the one or more internal standards are selected from the lipid class of wax esters (WE), and the internal standards are selected from the group consisting of WE (FA19:1/OH8:0) and WE (FA17:1/OH8:0). Additionally, with regard to this feature, at least one of the one or more internal standards is isotopically labeled.

In an additional feature, the sample is a sebum sample and is collected using sebum tape, swabs, or filter paper. In a further feature, the method further comprises determining the amount of cholesterol. In another feature, the method further comprises determining the presence, absence or amount of one or more fatty acid isomers in the sample. In yet another feature, the samples are injected directly into the mass spectrometer without a prior separation or purification step.

In another aspect of the invention, a kit comprises one or more internal standards for each of one or more lipid classes selected from the group consisting of wax esters (WE), squalene (SQ), triacylglycrides (TAG), diacylglycerols (DAG), free fatty acids (FFA), cholesterol (CH), cholesteryl esters (CE) and combinations thereof, and packaging material and instructions for using the kit. In a feature of this aspect, at least one of the one or more internal standards is isotopically labeled.

In a further aspect of the invention, a kit comprises one or more internal standards for each of one or more lipid classes selected from the group consisting of wax esters (WE), squalene (SQ), triacylglycrides (TAG), diacylglycerols (DAG), free fatty acids (FFA), cholesterol (CH), cholesteryl esters (CE) and combinations thereof, and for each of one or more tocopherols (TOC), selected from the group consisting of alpha-tocopherol, beta-tocopherol, gamma tocopherol, and delta-tocopherol, and combinations thereof, and packaging material and instructions for using the kit. In a feature of this aspect, at least one of the one or more internal standards is isotopically labeled.

In an additional aspect of the invention, a method for determining in a sample, by mass spectrometry, the presence, absence or amount of one or more lipid species selected from the group consisting of squalene (SQ), free fatty acids (FFA), complex lipids, and combinations thereof, comprising multiple steps. The steps include subjecting the sample to an ionization source under conditions suitable to produce one or more ions detectable by mass spectrometry from each of the one or more of the lipid species; measuring, by mass spectrometry, the amount of the one or more ions from each of the one or more lipid species; and using the measured amount of the one or more ions to determine the amount of each of the one or more lipid species in the sample.

In a feature of this aspect, the one or more complex lipids are selected from the group consisting of WE, TAG, DAG, and CE. In a further feature, the method further comprises determining the number of carbons and double bonds of one or more fatty acids of the one or more complex lipid species. In an additional feature, the sample is a sebum sample or a sebocyte sample. When the sample is a sebum sample, it may be collected using sebum tape, swabs, or filter paper. In another feature, the amount of the one or more lipid species from the one or more lipid classes are determined from a single injection. In a still further feature, the method further comprises determining the amount of cholesterol. In another feature, the samples are injected directly into the mass spectrometer without a prior separation or purification step.

In an additional aspect of the invention, a method to detect the presence, absence or amount of one or more lipid species in a sample, wherein the lipid species are classified within the lipid classes consisting of SQ, WE, DAG, TAG, FFA, cholesterol, and CE, comprises injecting a single injection of a sample extract into a mass spectrometer without a prior separation or purification step; and using the mass spectrometer, determining the identity of the one or more lipid species, and the concentration of the one or more lipid species in the sample.

In a further aspect of the invention, a method to detect the presence, absence or amount of one or more lipid species and one or more TOCs in a sample, wherein the lipid species are selected from the lipid classes consisting of SQ, WE, DAG, TAG, FFA, cholesterol, and CE, and the one or more TOCs are selected from the group consisting of alpha-tocopherol, beta-tocopherol, gamma tocopherol, and delta-tocopherol, comprises injecting a single injection of a sample extract into a mass spectrometer without a prior separation or purification step and using the mass spectrometer, determining the identity of the one or more lipid species, the identity of the one or more TOCs, the concentration of the one or more lipid species, and the concentration of the one or more TOCs in the sample.

In a feature of this aspect, the presence, absence or amount of the one or more fatty acid isomers in the sample is determined using GC-FAME (gas chromatography-fatty acid methyl ester) analysis.

### BRIEF DESCRIPTION OF THE DRAWINGS

FIG. 1 is a chart showing the R² values from a linearity study of lipid species within the indicated lipid classes.

FIG. 2 is a chart showing the percent fatty acid composition (across all lipid classes) for sebum samples analyzed with the methods described in Example 1 compared to a standard analysis method (FAME-GC/MS analysis).

FIG. 3 is a graph showing the concentrations of 298 WE lipid species measured in a sebum sample using the methods described herein. The species are on the X-axis and the concentration (in nmol/tape) is shown on the Y-axis.

FIG. 4 is a graph showing the concentrations of the 575 TAG lipid species measured in a sebum sample using the methods described herein. The species are on the X-axis and the concentration (in nmol/tape) is shown on the Y-axis.

FIG. 5 is a table showing a matrix of the concentrations of the 47 DAG lipid species measured in a sebum sample using the methods described herein. Each of the theoretical combinations of two fatty acids is indicated, with one fatty acid listed in columns and a second fatty acid in rows. The amount of the lipid species measured in the sample is indicated with the concentration (in nmol/tape) of the DAG lipid species in the corresponding box.

### DETAILED DESCRIPTION

Methods are described for measuring the presence, absence, or amount of one or more lipid species from one or more lipid classes in a sample by mass spectrometry. In some embodiments the lipid species may be selected from the lipid classes consisting of squalene (SQ), wax esters (WE), free fatty acids (FFA), triacylglycerols (TAG), diacylglycerols (DAG), cholesteryl esters (CE), cholesterol (CH), and combinations thereof. Mass spectrometric methods are described for quantifying lipid species from one or more lipid classes in a sample using a single injection method. Additionally, the described methods can be used to determine the number of carbons and double bonds in one or more constituent fatty acids (i.e, fatty acid composition) of the one or more lipid species. The methods may be performed without a prior separation or purification step. In an example, the methods may be performed without a chromatography step.

In other embodiments, methods are described for measuring the presence, absence, or amount of one or more lipid species from one or more lipid classes selected from the lipid classes consisting of squalene (SQ), wax esters (WE), free fatty acids (FFA), triacylglycerols (TAG), diacylglycerols (DAG), cholesteryl esters (CE), cholesterol (CH), and one or more tocopherols (TOC) in a sample by mass spectrometry. The tocopherols may be selected from α-tocopherol, β\γ-tocopherol, δ-tocopherol, and combinations thereof.

The described methods can simultaneously quantify and resolve the molecular composition of major lipid classes of sebum. As will be described more fully below, sebum samples collected from healthy volunteers were subjected to organic solvent extraction followed by automated flow injection into a mass spectrometer (referred to herein as flow injection analysis-mass spectrometry or FIA-MS) operated in Multiple Reaction Monitoring (MRM) mode. Starting from a broad combinatorial list of greater than 2,500 molecular species, approximately 1,000 specific lipid species were identified. The identified species reproducibly account for the vast majority of total signal in each lipid class. Further, at least one internal standard per class was included. The analytical reproducibility, recovery, and linearity of the assay were validated. Together, these optimized, high-throughput methods provide unprecedented insight into the lipidomic composition of sebum.

Using the described methods, over 2,500 lipid species from a plurality of lipid classes, including squalene, and wax esters, as well as diacylglycerols, triacylglycerols, cholesteryl esters, cholesterol, free fatty acids, and one or more lipid-soluble tocopherols or total tocopherol in a sebum sample can be determined by mass spectrometry analysis alone, without the use of a prior purification step, for example, without chromatographic separation. The ability to measure, in a single injection and in the absence of chromatography or purification, a plurality of lipid molecules and total tocopherol or one or more tocopherols in various combinations, reduces the time required to obtain analysis results and uses fewer resources in terms of laboratory disposables (e.g., organic solvents, tubes, pipette tips, reagents), laboratory instruments and human resources. These improvements lead to savings by decreasing the costs of the assays and increasing the instrument and laboratory capacity for sample analysis.

The described methods can be used in combination with GC-FAME analysis, which can distinguish isomeric species of fatty acids, such as straight-chain and branched-chain isomers of the same fatty acid. Fatty acids detected using GC-FAME are quantified using calibration curves. The lipids are broken down prior to GC-FAME analysis, so the assay does not measure individual complex lipid species or show how much each fatty acid contributed to the composition of each lipid class. Instead, the GC-FAME analysis reports the total fatty acid composition of the sebum. When used together, FIA-MS and GC-FAME assays provide complementary information that enables a comprehensive characterization of the sebum lipidome.

Prior to describing this invention in further detail, the following terms are defined.

### Definitions

“Lipids” or “lipid molecules” refers to organic small molecules that are insoluble in water or other polar solvent but are soluble in non-polar solvents (e.g., ether). Lipids are structurally diverse molecules with biological functions that include cellular signaling, energy storage, and providing structural components of cellular membranes. Non-limiting examples of lipids include squalene (SQ), fatty acids (FA), wax esters (WE), cholesterol (CH), cholesterol esters (CE), triacylglycerol, (TAG), and diacylglycerols (DAG). Some lipids are comprised of a single structure (i.e., consist of only one structural component) and are referred to herein as “simple lipids”. Non-limiting examples of so-called simple lipids are SQ and CH. Some lipids are comprised of a plurality of structural components, including one or more fatty acids, and are referred to herein as “complex lipids” (CL). DAG, TAG, CE, and WE are non-limiting examples of complex lipids. As used herein, a “lipid species” refers to an individual lipid molecule that is defined to the level of the chemical formula (e.g., SQ, CH, FFA (16:0)), or, for complex lipids (CL) which are comprised of one or more fatty acids or a combination of fatty acids, defined to identify the number of carbons and double bonds in at least one constituent fatty acid (e.g., WE (FA19:1/OH8:0), DAG (16:0/16:0), DAG (18:0/18:4), TAG (39:0-FA12:0), etc.).

“Lipid class” as used herein refers to lipid molecules that have structural similarity, and are therefore grouped together as a class. As used herein, non-limiting examples of lipid classes include squalene (SQ), wax esters (WE), cholesterol (CH), free fatty acids (FFA), cholestryl/cholesterol esters (CE), triacylglycerols (TAG), and diacylglycerols (DAG). Some classes include only a single lipid species. For example, squalene (SQ) is the single lipid species in the SQ class. Other classes include a plurality of lipid species. For example, the free fatty acid class (FFA) includes a plurality of lipid species (e.g., FFA (14:0), FFA (16:0), FFA (16:1), and FFA (18:0)). Yet other lipid classes include a plurality of lipid species that includes complex lipids. For a sample, the concentration of each fatty acid across one or more classes of complex lipids (CL) can be determined by summing the concentration of each complex lipid that contains that fatty acid. This value is referred to as the “CL fatty acid concentration”. Additionally, the percent contribution of each fatty acid across one or more classes of complex lipids can be determined. This value is referred to as the “CL fatty acid compositions”.

“Lipidome” refers to the complete lipid profile within a cell, tissue, biological fluid or organism. The lipid profile is comprised of lipids in multiple, distinct structural lipid classes. As used herein, “Sebum lipidome” refers to the lipids in the plurality of lipid classes present in sebum or a sebum sample.

The term “chromatography” refers to a physical method of separation in which the components (i.e., chemical constituents) to be separated are distributed between two phases, one of which is stationary (stationary phase) while the other (the mobile phase) moves in a definite direction. The mobile phase may be gas (“gas chromatography”, “GC”) or liquid (“liquid chromatography”, “LC”; “Thin-layer chromatography”, “TLC”).

The term “Mass Spectrometry” (MS) refers to a technique for measuring and analyzing molecules that involves ionizing or ionizing and fragmenting a target molecule, then analyzing the ions, based on their mass/charge ratios, to produce a mass spectrum that serves as a “molecular fingerprint”. There are several commonly used methods to determine the mass to charge ratio of an ion, some measuring the interaction of the ion trajectory with electromagnetic waves, others measuring the time an ion takes to travel a given distance, or a combination of both. The data from these fragment mass measurements can be searched against databases to obtain identifications of target molecules.

The terms “operating in negative mode” or “operating in negative ionization mode” refer to those mass spectrometry methods where negative ions are generated and detected. The terms “operating in positive mode” or “operating in positive ionization mode” refer to those mass spectrometry methods where positive ions are generated and detected.

The term “mass analyzer” refers to a device in a mass spectrometer that separates a mixture of ions by their mass-to-charge (“m/z”) ratios.

The term “m/z” refers to the dimensionless quantity formed by dividing the mass number of an ion by its charge number. It has long been called the “mass-to-charge” ratio.

As used herein, the term “source” or “ionization source” refers to a device in a mass spectrometer that ionizes a sample to be analyzed. Examples of ionization sources include electrospray ionization (ESI), atmospheric pressure chemical ionization (APCI), heated electrospray ionization (HESI), atmospheric pressure photoionization (APPI), flame ionization detector (FID), matrix-assisted laser desorption ionization (MALDI), etc.

As used herein, the term “detector” refers to a device in a mass spectrometer that detects ions.

The term “ion” refers to any object containing a charge, which can be formed for example by adding electrons to or removing electrons from the object.

The term “mass spectrum” refers to a plot of data produced by a mass spectrometer, typically containing m/z values on x-axis and intensity values on y-axis.

The term “tandem MS” refers to an operation involving multiple stages of MS selection with fragmentation occurring between the stages. In a first MS stage, ions are formed in the source. Ions of a particular mass-to-charge ratio, each representing one (and possibly more than one) chemical constituent, are selected, and fragment ions are created. The resulting ions are then separated and detected in a second stage of mass spectrometry. The ion of interest in the first MS stage corresponds to a “parent” or precursor ion, while the ions created during the second MS stage(s) correspond to sub-components of the parent ion and are herein referred to as “daughter” or “product” ions.

Thus, tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion. Tandem MS may be repeated on daughter ions to determine “grand-daughter” ions, for example. Thus, tandem MS is not limited to two-levels of fragmentation, but is used generically to refer to multi-level MS, also referred to as “MSⁿ”. The term “MS/MS” is a synonym for “MS²”. For simplicity, the term “daughter ion” hereinafter refers to any ion created by a secondary or higher-order (i.e., not the first) MS.

The “amount” of one or more lipid molecules means the chemical or mass concentration of the lipid molecule measured in the sample. For example, the amount or concentration may be expressed as the molar concentration, mass fraction, mole fraction, molality, percentage.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological fluid or tissue such as, for example, sebum, cells (e.g., sebocyte cells) blood, blood plasma (plasma), blood serum (serum), urine, cerebral spinal fluid (CSF), or tissue.

“Subject” means any animal, but is preferably a mammal, such as, for example, a human, monkey, mouse, rabbit or rat.

### I. Sample Preparation and Quality Control (QC)

Sample extracts are prepared by partitioning the lipids from other molecules (e.g., proteins, nucleic acids, other small molecule metabolites) that may be present in the sample. In one example, the sample is a sebum sample, and samples are collected using sebum tape (e.g., sebutape), swabs (e.g. cotton swabs), or filter paper. In another example, the sample is sebocyte cells. Lipid molecules may be extracted from samples using methods known to one of ordinary skill in the art, for example, by using methanol. Some or all lipid molecules in a sample may be bound to proteins. Various methods may be used to disrupt the interaction between lipid molecules and protein prior to MS analysis. For example, the lipid molecules can be extracted from a sample to produce a liquid extract, while the proteins that are present can be precipitated. Proteins can be precipitated using, for example, a solution of methanol or ethyl acetate. In an exemplary method, to precipitate the proteins in the sample, a methanol or ethyl acetate solution is added to the sample, then the mixture may be spun in a centrifuge or centrifuge filtered to separate the liquid supernatant, which contains the extracted lipid molecules, from the precipitated proteins.

In other embodiments, lipid molecules may be released from protein without precipitating the protein. For example, a formic acid solution may be added to the sample to disrupt the interaction between protein and lipid molecule. Alternatively, ammonium acetate, ammonium sulfate, a solution of formic acid in ethanol, or a solution of formic acid in methanol may be added to the sample to disrupt ionic interactions between protein and lipid molecule without precipitating the protein.

In some embodiments, the sample extract may be directly injected into the mass spectrometer without the prior use of chromatography such as liquid chromatography, gas chromatography, or thin layer chromatography to purify or enrich the amount of the lipid molecules.

In some embodiments, the extracted sample may be divided such that a portion is used for FIA-MS analysis and another portion is used for GC-FAME analysis.

To assess, for example, precision, accuracy, calibration range, or analytical sensitivity of methods of detecting and quantifying lipid molecules, quality control (QC) samples may be used. Such QC samples are subjected to the same extraction methods as the experimental samples.

### II. Mass Spectrometry and Quantitation

One or more lipid species from one or more lipid classes may be detected by mass spectrometry. Mass spectrometry is performed using a mass spectrometer that includes an ion source for ionizing the sample and creating charged molecules for further analysis. Ionization of the sample may be performed by, for example, electrospray ionization (ESI). Other ion sources may include, for example, atmospheric pressure chemical ionization (APCI), heated electrospray ionization (HESI), atmospheric pressure photoionization (APPI), flame ionization detector (FID), or matrix-assisted laser desorption ionization (MALDI). The choice of ionization method may be determined based on a number of considerations. Exemplary considerations include the lipid molecule to be measured, type of sample, type of detector, and the choice of positive or negative mode.

After a sample has been ionized, the positively or negatively charged ions may be analyzed to determine a mass-to-charge ratio. Exemplary suitable analyzers for determining mass-to-charge ratios include quadrupole analyzers, ion trap analyzers, and time of flight analyzers. The ions may be detected using, for example, a selective mode or a scanning mode. Exemplary scanning modes include multiple reaction monitoring (MRM) and selected reaction monitoring (SRM).

The sample extract may be injected directly into the ionization source of the mass spectrometer. The sample may be injected at a flow rate of about 5 μl/min to about 10 μl/min. The run time may be less than 7 minutes, and the total time between sample injections may be less than 14 minutes.

The one or more lipid species from one or more lipid classes and the one or more lipid-soluble tocopherols may be ionized in positive or negative mode to create one or more ions. For example, the lipid species from the lipid classes of squalene, wax esters, diacylglycerols, triacylglycerols, and cholesteryl esters, and the one or more tocopherols may be ionized in positive mode. In another example, the lipid species from the lipid class of free fatty acids may be ionized in negative mode. The lipid species and TOCs ionized in positive mode and the lipid species ionized in negative mode may be measured in a single injection of the sample extract.

In one example, mass spectrometry may be tandem MS and may be performed, for example, using AB Sciex QTrap 5500 tandem mass spectrometers. Tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion.

Mass spectrometer instrument settings may be optimized for the given method and/or for the particular mass spectrometer used. The instrument may use various gases, for example, nitrogen, helium, argon, or zero air. In one example, the mass spectrometer may be operated in positive ionization mode. The ionspray voltage setting may range from about 0.5 kV to about 5.0 kV; in one embodiment the voltage may be set at 4.1 kV. The source temperature may range from about 100° C. to about 600° C.; in one embodiment the source temperature may be set at 250° C. The curtain gas may range from about 10 to about 55 psi; in one embodiment the curtain gas is set at 17 psi. The nebulizer and desolvation gas flow rates may range from about 0 to about 90 psi. In one embodiment the nebulizer gas may be set at 17.0 psi and the desolvation gas may be set at 25.0 psi. The collisionally activated dissociation (CAD) gas setting may range from high to low; in one embodiment the CAD gas is set at medium. Declustering potential may range from about 15V to about 170V. The collision energy (CE) may range from about 10 eV to about 100 eV. The entrance potential (EP) setting may range from about 5V to about 30V. The collision cell exit potential (CXP) setting may range from about 8V to about 16V.

In another example, the instrument may be operated in negative ionization mode. Ionspray voltage settings may range from about −0.5 kV to about −5.5 kV; in one embodiment the voltage may be set at −2.5 kV. The source temperature may range from about 100° C. to about 600° C.; in one embodiment the source temperature may be set at 250° C. The curtain gas may range from about 10 to about 55 psi; in one embodiment the curtain gas may be set at 17.0 psi. The nebulizer and desolvation gas flow rates may range from about 0 to about 90 psi. In one embodiment the nebulizer gas may be set at 17.0 psi and the desolvation gas may be set at 25.0 psi. The CAD gas may range from low to high. In one example the CAD may be set, for example, at medium. Declustering potential may range from about −30V to about −10V. The collision energy (CE) may range from about −30 eV to about −5 eV. The entrance potential (EP) setting may range from about −30V to about −5V. The collision cell exit potential (CXP) setting may range from about −20V to about −8V.

In one example, MS may be accurate-mass MS. For example, the accurate-mass mass spectrometry may use a quadrupole time-of-flight (Q-TOF) analyzer. In exemplary embodiments, accurate-mass MS may be accurate-mass tandem MS.

The mass spectrometer typically provides the user with an ion scan (i.e., a relative abundance of each ion with a particular mass/charge over a given range of timepoints). Mass spectrometry data may be related to the amount of the lipid molecule or TOC molecule in the original sample by a number of methods. In one example, an internal standard (IS) may be used. Internal standards may be added to test samples and to quality control samples for quantitation of individual TOCs or lipid species. At least one internal standard for each lipid class to be measured may be used. The ratio of TOC or lipid molecule ion intensity to internal standard ion intensity in the samples, along with the known concentration of the internal standard, can be used for quantitation. One or more internal standards, from one or more TOCs and one or more lipid classes selected from TAG, DAG, FFA, SQ, WE, CE, and CH may be used for quantitation. In some embodiments, the internal standards may be isotopically labeled using, for example, deuterium (²H, denoted “d”), ¹³C, or ¹⁵N isotopes. Any atom or any number of atoms of the internal standard may be labeled with the isotope. In some embodiments, all of the IS may be isotopically labeled. In some embodiments, none of the IS are isotopically labeled. In other embodiments a combination of isotopically labeled IS and unlabeled IS may be used. In one example, the internal standards TAG (16:0-d9/16:0/18:1), TAG (16:0-d9/18:0/18:1), TAG (16:0-d9/18:1/18:1), TAG (16:0-d9/18:2/18:1), TAG (16:0-d9/18:3/18:1), TAG (16:0-d9/20:3/18:1), TAG (16:0-d9/20:4/18:1), and/or TAG (16:0-d9/22:6/18:1) may be used for the quantitation of triacylglycerol lipid species; the internal standards DAG (16:0-d9/16:0), DAG (16:0-d9/18:0), DAG (16:0-d9/18:1), DAG (16:0-d9/18:2), DAG (16:0-d9/18:3), DAG (16:0-d9/20:4), DAG (16:0-d9/20:5), and/or DAG (16:0-d9/22:6) may be used for the quantitation of diacylglycerol lipid species; the internal standards FFA (16:0)-d9, FFA (18:1)-d17 and/or FFA (17:1) may be used for the quantitation of free fatty acids; the internal standard SQ-d6 may be used for the quantitation of SQ; the internal standards WE (FA19:1/OH8:0), WE (FA17:1/OH8:0), WE (FA16:0-d9/OH16:0d37), and/or WE (FA16:1/OH18:0-d37) may be used for the quantitation of WE lipid species; the internal standards CE (16:0)-d7, CE (16:1)-d7, CE (18:1)-d7, CE (18:2)-d7, CE (20:3)-d7, CE (20:4)-d7, CE (20:5)-d7, and/or CE (22:6)-d7 may be used for the quantitation of cholesterol ester lipid species; the internal standard cholesterol-d7 may be used for the quantitation of cholesterol; and/or the internal standards α-tocopherol-d₆, α-tocopherol-¹³C₆, α-tocopherol-¹³C₃, α-tocopherol-¹³C₉, may be used for the quantitation of tocopherols.

A calibration standard may also be used for quantitation. A calibration standard is used to generate a standard curve (calibration curve) so that the relative abundance of a given ion may be converted into an absolute amount of the analyte, such as a TOC or lipid molecule. An internal standard may be added to calibration standards. In another example, the calibration standard may be an external standard and a standard curve may be generated based on ions generated from those standards to calculate the quantity of one more TOC or lipid molecules. In a further example, the external standard may be an unlabeled TOC or lipid molecule.

The analysis data may be sent to a computer and processed using computer software. In one example, each individual TOC or lipid species may be quantified based on the ratio of signal intensity for target compounds to the signal intensity for an assigned internal standard of known concentration. Total TOC concentrations may be calculated from the sum of each TOC detected in the sample. Lipid species compositions may be determined by calculating the proportion of individual lipid species within each lipid class. Lipid class concentrations may be calculated from the sum of all lipid species within a lipid class, and lipid class compositions may be determined by calculating the proportion of lipid classes within the sample. The fatty acid concentrations may be calculated from the sum of all lipid species within a lipid class containing a specific fatty acid, and fatty acid compositions may be determined by calculating the proportion of individual fatty acids within each lipid class.

### III. Kit

A kit for assaying one or more TOCs and/or one or more lipid species from one or more lipid classes selected from the group consisting of WE, SQ, TAG, DAG, FFA, CE, CH, and combinations thereof is described herein. For example, a kit may include packaging material, one or more control samples, sample collection receptacles, and measured amounts of one or more internal standards in quantities sufficient for one or more assays. Additional kit components in separate packaging could include buffers and other reagents for the detection and/or quantification of lipid molecules in a sample of interest. Kits may also comprise instructions recorded in tangible form (e.g. on paper such as, for example, an instruction booklet or an electronic medium) for using the reagents to measure the one or more lipid species.

In other embodiments, a kit for assaying one or more lipid species from one or more lipid classes selected from the group consisting of WE, SQ, TAG, DAG, FFA, CE, CH, and combinations thereof and one or more TOCs selected from the group consisting of alpha-tocopherol, beta-tocopherol, gamma-tocopherol, and delta-tocopherol is described herein.

### EXAMPLES

### Reagents

HPLC grade (99.9%) methanol was obtained from JT Baker; HPLC grade (99.9%) dichloromethane was obtained from Honeywell Burdick & Jackson; ammonium acetate (BioUltra≥99.0%). TAG (16:0-d9/16:0/18:1), TAG (16:0-d9/18:0/18:1), TAG (16:0-d9/18:1/18:1), TAG (16:0-d9/18:2/18:1), TAG (16:0-d9/18:3/18:1), TAG (16:0-d9/20:3/18:1), TAG (16:0-d9/20:4/18:1), TAG (16:0-d9/22:6/18:1), CE (16:0)-d7, CE (16:1)-d7, CE (18:1)-d7, CE (18:2)-d7, CE (20:3)-d7, CE (20:4)-d7, CE (20:5)-d7, and CE (22:6)-d7 were synthesized by Avanti Polar Lipids or Echelon Biosciences; DAG (16:0-d9/16:0), DAG (16:0-d9/18:0), DAG (16:0-d9/18:1), DAG (16:0-d9/18:2), DAG (16:0-d9/18:3), DAG (16:0-d9/20:4), DAG (16:0-d9/20:5), DAG (16:0-d9/22:6) were synthesized by Avanti Polar Lipids; FFA (16:0)-d9, FFA (17:1) were obtained from Avanti Polar Lipids; SQ-d6 was obtained from Toronto Research Chemicals, and α-tocopherol-d₆ was obtained from Santa Cruz Biotechnology. WE (FA19:1/OH8:0) was synthesized in-house.

### GC-FAME Analysis

The total fatty acid content, in nmol/tape, of the methyl esters of the 32 fatty acids shown in Table 1 was determined using GC-FAME. Samples of the 32 fatty acids were provided, and isotopically-labeled internal standards were added to each of the samples. The samples were evaporated to dryness under a stream of nitrogen. The dried lipid extract was subjected to methylation with methanol/sulfuric acid for one hour at 90° C. resulting in the formation of the corresponding methyl esters of free fatty acids and conjugated fatty acids. The reaction mixture was neutralized with potassium carbonate and extracted with hexanes. An aliquot of the hexanes layer was removed and injected onto a 7890A/5975C GC/MS system (Agilent Technologies, CA) equipped with a DB-225 column (Agilent Technologies, CA) using hydrogen as the carrier gas. Mass spectrometric analysis was performed in the single ion monitoring (SIM) positive mode with electron ionization. Quantitation was performed using linear or quadratic regression analysis generated from fortified calibration standards.

TABLE 1
Fatty Acid Concentration as Measured Using GC-FAME

| | Concentration (nmol/tape) | | | | |
|---|---|---|---|---|---|
| Fatty Acid | Sample 1 | Sample 2 | Sample 3 | Sample 4 | Sample 5 |
| 12:0-iso | 0.3176 | 0.5720 | 1.4959 | 0.2991 | 5.6294 |
| 12:0 | 11.6290 | 12.2219 | 20.8878 | 23.4857 | 21.8632 |
| 13:0-iso | 0.1300 | 0.0784 | 0.2296 | 0.1772 | 0.4118 |
| 13:0-anteiso | 1.6533 | 1.0576 | 2.7471 | 2.6494 | 6.7492 |
| 13:0 | 3.3102 | 1.3883 | 2.4776 | 3.3866 | 4.4359 |
| 14:0-iso | 2.7794 | 3.6912 | 9.8387 | 1.8896 | 34.4164 |
| 14:0 | 90.2949 | 42.7516 | 80.3700 | 103.8759 | 136.1017 |
| 15:0-iso | 3.4156 | 1.6113 | 3.6155 | 3.2506 | 6.6492 |
| 15:0-anteiso | 10.9306 | 4.5808 | 12.9125 | 12.5540 | 32.1618 |
| 15:0 | 42.6142 | 11.7618 | 23.6052 | 36.7631 | 53.2455 |
| 16:0-iso | 2.7539 | 1.7236 | 4.2862 | 1.6584 | 9.8103 |
| 16:0 | 175.6172 | 41.7769 | 71.3177 | 131.7829 | 128.3999 |
| 16:1n10 | 216.8009 | 40.3882 | 64.0423 | 149.2054 | 129.6340 |
| 16:1n7 | 0.4847 | 0.2228 | 0.4803 | 0.3828 | 0.2493 |
| 17:0-anteiso | 1.8927 | 0.4964 | 1.2371 | 1.5392 | 2.0405 |
| 17:0 | 7.6904 | 1.9935 | 2.8384 | 5.7555 | 4.4932 |
| 18:0-iso | 0.2884 | 0.1313 | 0.3646 | 0.1336 | 0.9425 |
| 18:0 | 29.7959 | 16.9584 | 29.8490 | 32.2572 | 26.4892 |
| 18:1n10/ | 88.8585 | 18.6842 | 28.7490 | 54.5466 | 64.9544 |
| 18:1n9 | | | | | |
| 18:1n7 | 0.7800 | 0.2813 | 0.4948 | 0.3833 | 0.3815 |
| 18:2n10 | 8.0087 | 2.7835 | 5.2824 | 9.4361 | 9.8939 |
| 18:2n6 | 11.7708 | 5.3710 | 9.7848 | 10.0977 | 9.9225 |
| 19:0-anteiso | 0.2022 | 0.0354 | 0.0958 | 0.1042 | 0.1984 |
| 18:3n3 | 20.8117 | 5.9578 | 8.4398 | 15.8063 | 16.2763 |
| 20:0-iso | 0.2466 | 0.0903 | 0.2601 | 0.1036 | 0.7679 |
| 20:0 | 3.1937 | 0.7584 | 1.1257 | 1.9717 | 2.9867 |
| 21:0-iso | 0.0562 | 0.0136 | 0.0280 | 0.0309 | 0.0598 |
| 21:0-anteiso | 0.4146 | 0.0629 | 0.1655 | 0.2257 | 0.4170 |
| 21:0 | 0.3684 | 0.1381 | 0.1608 | 0.3290 | 0.3321 |
| 22:0 | 2.1376 | 0.7176 | 0.9739 | 1.7083 | 2.0855 |
| 23:0 | 0.8563 | 0.2999 | 0.3900 | 0.7962 | 0.7310 |
| 24:0 | 5.1927 | 1.4202 | 1.8774 | 3.9540 | 4.9102 |

### Example 1: Mass Spectrometry Method for Quantitative Sebum Lipid and TOC Assays

A. Sample Preparation

Sebum samples were collected from volunteer subjects using sebum tapes. Sample preparation was carried out in glass sample tubes. Lipid molecules were extracted using methanol. Following addition of 3 ml of methanol, the sebum tape samples were vortexed, then incubated for 10 min at room temperature. After samples were vortexed, an aliquot was removed for optional FAME-GC/MS analysis and another aliquot was taken from each study sample and pooled to form a single pooled sample that was used for quality control. To each sample tube, 75 μL of a working internal standard (WIS) solution of dichloromethane (DCM)/methanol (50/50) containing the appropriate internal standard(s) was added. The WIS solution contained one or more internal standards for each lipid class and one for TOCs. The internal standards are listed in Table 2. The sample blanks were extracted by adding 75 μL of DCM/methanol (50/50) without internal standards. Samples were vortexed, and the sebum tapes were removed from the tubes. Samples were dried and then reconstituted in 300 μL DCM/methanol (90/10)+10 mM ammonium acetate. Samples were vortexed, centrifuge filtered, and transferred to vials for mass spectrometry analysis. The WIS concentrations for each of the lipid classes and TOCs are shown in Table 2. The percentage for each internal standard refers to percent by weight. All WIS solutions were prepared in a solution of DCM/methanol (50/50).

TABLE 2
Working Internal Standard (WIS) Solutions

| Internal | | |
|---|---|---|
| Standard | | Concentration |
| Class | Internal Standard | (mg/mL) |
| dTAG | TAG(16:0-d9/16:0/18:1) (20%) | 0.151 |
| | TAG(16:0-d9/18:0/18:1) (20%) | |
| | TAG(16:0-d9/18:1/18:1) (20%) | |
| | TAG(16:0-d9/18:2/18:1) (20%) | |
| | TAG(16:0-d9/18:3/18:1) (5%) | |
| | TAG(16:0-d9/20:3/18:1) (5%) | |
| | TAG(16:0-d9/20:4/18:1) (5%) | |
| | TAG(16:0-d9/22:6/18:1) (5%) | |
| dFFA | FFA(16:0)-d9 (50%) | 0.108 |
| | FFA(17:1) (50%) | |
| dDAG | DAG(16:0-d9/16:0) (20%) | 0.0317 |
| | DAG(16:0-d9/18:0) (20%) | |
| | DAG(16:0-d9/18:1) (20%) | |
| | DAG(16:0-d9/18:2) (20%) | |
| | DAG(16:0-d9/18:3) (5%) | |
| | DAG(16:0-d9/20:4) (5%) | |
| | DAG(16:0-d9/20:5) (5%) | |
| | DAG(16:0-d9/22:6) (5%) | |
| dSQ | SQ-d6 | 0.265 |
| WE-IS | WE(FA19:1/OH8:0) | 0.0185 |
| dCE | CE(16:0)-d7 (5%) | 0.0265 |
| | CE(16:1)-d7 (5%) | |
| | CE(18:1)-d7 (20%) | |
| | CE(18:2)-d7 (50%) | |
| | CE(20:3)-d7 (5%) | |
| | CE(20:4)-d7 (5%) | |
| | CE(20:5)-d7 (5%) | |
| | CE(22:6)-d7 (5%) | |
| dTOC | α-tocopherol-d6 | 0.000437 |

B. MRM and Lipid Species Selection

In an initial step, the number of lipid species that could possibly be present in a sebum sample was calculated. This calculation was based on the fatty acids and lipid classes described in the literature to be present in sebum samples, and a list of all possible lipid species within the SQ, WE, TAG, DAG, FFA, and CE lipid classes was generated. From this analysis, it was determined that there are over 2,500 possible lipid species in sebum samples.

Chemical standards from the lipid-soluble TOCs and from the WE, TAG, DAG, FFA, and CE lipid classes were analyzed by mass spectrometry to determine the MRM pairs useful for monitoring the TOCs and each of the over 2,500 lipid species in a sebum sample. Based on this analysis, the MRM pair used to monitor each TOC and each lipid species was selected. One exemplary parent and daughter ion pair (representing one MRM pair) was selected per lipid species in the lipid classes WE, TAG, DAG, FFA, and CE. One exemplary parent and daughter ion pair was also selected for the tocopherols α-tocopherol, δ-tocopherol, and the combination of β- and γ-tocopherol (β/γ-tocopherol). One MRM was used for β/γ-tocopherol because both the parent ions and the major daughter ions of β-tocopherol and γ-tocopherol have identical m/z and cannot be distinguished from each other using the described methods. The most abundant ions were selected for monitoring. Mass spectrometry analysis of SQ resulted in one parent ion and two daughter ions; the daughter ion with the lowest background was selected for monitoring.

The selected MRMs were then used to measure the amount of lipid species and TOCs using sebum samples. Three injections of the sample extract into the mass spectrometer were required to measure the amount of the over 2,500 lipid species in the samples using these MRMs. Analysis of the data of the measured amounts of the more than 2,500 lipid species in the samples showed that by selecting the most abundant lipid species in the samples, over 90% of the lipid molecules in a sebum sample could be measured in a single injection. Therefore, MRMs for the most abundant lipid species from the WE, TAG, and DAG lipid classes were selected for inclusion in the single injection analysis method along with MRMs for SQ and FFAs. For CEs, MRMs for 26 common lipid species were selected. For TOCs, one MRM for each of α-tocopherol, β/γ-tocopherol, and δ-tocopherol was selected. In this example, MRMs for 965 (of the over 2,500) lipid species and 3 TOCs were selected for inclusion in the single injection analysis method. MRMs were selected for 298 WE species, 47 DAG species, 575 TAG species, 18 FFAs, 26 CEs, and SQ as well as for the 3 TOCs.

C. Mass Spectrometry Method for Quantitative Detection of Lipid Species and Lipid-Soluble Tocopherols

An MS/MS method was developed to detect, in the same (single) injection of a sample, the levels of TOC and lipid species from the lipid classes consisting of WE, SQ, TAG, DAG, FFA, and CE.

Mass spectrometry was performed on the sample extracts using an AB SciEx QTrap 5500 mass spectrometer with Turbo V source (ESI). The sample extract was directly (i.e., without chromatographic separation) introduced into the ionization source of the mass spectrometer at a flow rate of 7 μl/min. The instrument was operated in multiple reaction monitoring (MRM) mode using 16 cycles with 20.0 msec per MRM, a settling time of 50 msec, and a pause between mass ranges of 5 msec. Each cycle of the instrument included a positive ionization mode and a negative ionization mode. The instrument was operated in positive ionization mode with ionspray voltage set at 4.1 kV and in negative ionization mode with ionspray voltage set at −2.5 kV. Source temperature was set at 250° C., curtain gas (e.g., nitrogen) at 17.0 psi, nebulizer gas (e.g., nitrogen) at 17.0 psi, desolvation gas (e.g., nitrogen) at 25.0 psi, and collisionally activated dissociation (CAD) gas (e.g., nitrogen) at medium. The run time was less than 7 minutes, and the total time from one sample injection to the next was 13.25 min.

Raw data were acquired from the instrument and processed using in-house software. For quantitation, a known concentration of internal standard provided single-point calibration for each lipid species. Lipid species (analytes) were quantitated as follows and values were reported in μM (IS refers to internal standard):

$$\frac{Analyte intensity}{IS intensity} \times IS concentration ( nmol / mL ) \times \frac{IS volume Added ( mL )}{Sample Weight ( g )}$$

MRMs were monitored for the following lipid species: SQ, 18 FFAs, 298 WEs, 47 DAGs, 575 TAGs, 26 CEs, and 3 TOCs.

### Example 2: Method Validation (Five Lipid Classes)

The precision of the method for measuring one or more lipid species in one or more lipid classes was evaluated using pooled sebum sample extract. Four samples were analyzed with two technical replicates per sample for a total of 8 samples. The precision was less than 5.2% RSD for lipid class concentrations, less than 8.0% RSD for lipid species concentrations, and less than 7.3% RSD for lipid species compositions. The results are presented in Table 3. Precision was also evaluated for fatty acid concentrations and compositions, and the results are presented in Table 4.

TABLE 3
Precision in sebum (by lipid class).

| | | | Lipid Species |
|---|---|---|---|
| | Lipid Class | Lipid Species | Compositions |
| Lipid | Concentrations | Concentrations | Median |
| Class | % RSD | Median % RSD | % RSD |
| TAG | 5.1 | 6.17 | 4.25 |
| DAG | 2.41 | 4.69 | 4.07 |
| FFA | 3.24 | 7.97 | 7.2 |
| WE | 1.88 | 5.05 | 4.72 |
| SQ | 2.27 | 2.27 | N/A |
| All | N/A | 5.82 | 4.42 |

TABLE 4
Precision of fatty acid concentrations and compositions in sebum.

| | Fatty Acid Concentrations | | Fatty Acid | |
|---|---|---|---|---|
| | Avg. | | Compositions | |
| Fatty Acid | concentration | % RSD | Avg. mol % | % RSD |
| Total [FA12:0] | 247.57 | 2.74 | 0.129 | 6.26 |
| Total [FA13:0] | 2512.56 | 4.64 | 1.305 | 2.99 |
| Total [FA14:0] | 10650.05 | 3.37 | 5.532 | 2.58 |
| Total [FA15:0] | 6152.21 | 2.51 | 3.198 | 3.38 |
| Total [FA16:0] | 41629.74 | 4.56 | 21.619 | 0.93 |
| Total [FA16:1] | 42737.87 | 5.67 | 22.183 | 1.12 |
| Total [FA17:0] | 5820.94 | 4.22 | 3.023 | 1.16 |
| Total [FA18:0] | 5003.23 | 6.55 | 2.595 | 2.37 |
| Total [FA18:1] | 24225.93 | 6.93 | 12.573 | 2.03 |
| Total [FA18:2] | 9442.65 | 4.13 | 4.907 | 3.27 |
| Total [FA19:0] | 7552.44 | 4.45 | 3.920 | 0.95 |
| Total [FA20:0] | 14202.70 | 8.38 | 7.369 | 4.49 |
| Total [FA21:0] | 8644.02 | 9.19 | 4.482 | 4.45 |
| Total [FA23:0] | 1456.02 | 16.41 | 0.761 | 19.96 |
| Total [FA18:3] | 5734.36 | 4.88 | 2.978 | 1.24 |
| Total [FA20:4] | 345.60 | 4.56 | 0.179 | 1.66 |
| Total [FA22:0] | 2110.91 | 6.90 | 1.095 | 1.96 |
| Total [FA22:6] | 383.04 | 5.82 | 0.199 | 2.29 |
| Total [FA24:0] | 2178.14 | 5.95 | 1.135 | 9.49 |
| Total [FA25:0] | 82.31 | 6.39 | 0.043 | 4.53 |
| Total [FA26:0] | 417.48 | 5.40 | 0.217 | 1.76 |
| Total [FA27:0] | 85.68 | 5.32 | 0.044 | 4.13 |
| Total [FA28:0] | 89.75 | 3.78 | 0.047 | 3.06 |
| Total [FA29:0] | 721.90 | 3.61 | 0.375 | 2.60 |
| Total [FA30:0] | 175.50 | 8.39 | 0.091 | 6.17 |

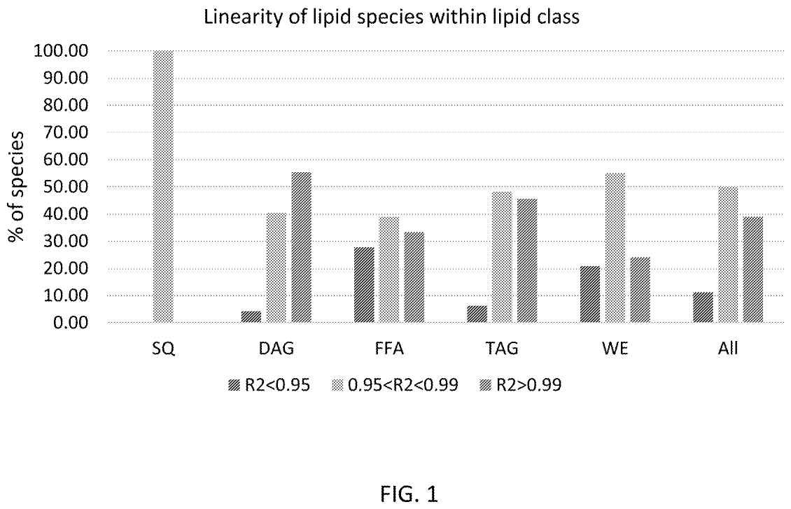

To assess linearity, a pooled sebum sample was diluted 0.111×, 0.222×, 0.333×, 0.444×, and 0.667×. Concentrations were calculated for each lipid species in each sample dilution to give an R² value for each lipid species. The results for the lipid species within each lipid class are presented in FIG. 1.

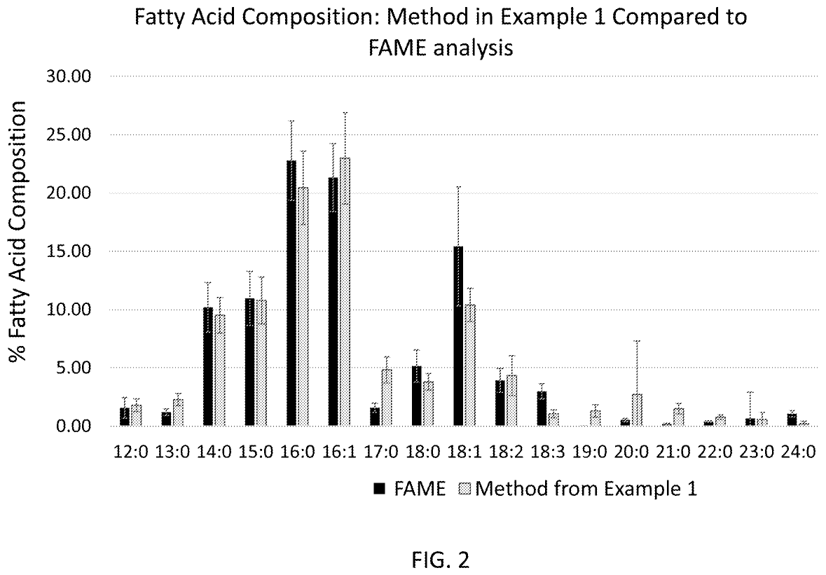

The method described in Example 1 (FIA-MS) was compared to the currently used FAME-GC/MS (FAME) analysis. Sebum samples collected from 20 subjects using sebum tape were used for the analysis. The total fatty acid composition of the samples was measured using both methods. A comparison of the fatty acid composition of 17 fatty acids, reported as % of total fatty acids, is shown in FIG. 2. The R² value for the linear regression of data generated using the two methods was 0.936, indicating that the fatty acid composition measured with each method was comparable. A comparison of fatty acid composition within lipid classes could not be made because FAME analysis reports the total fatty acid composition and cannot determine the fatty acid composition within the lipid class.

### Example 3: Lipid Analysis (Five Lipid Classes)

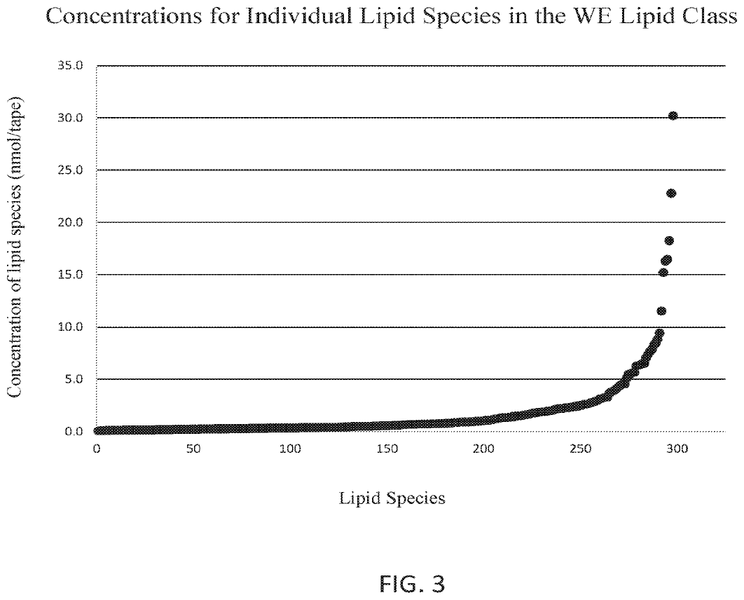

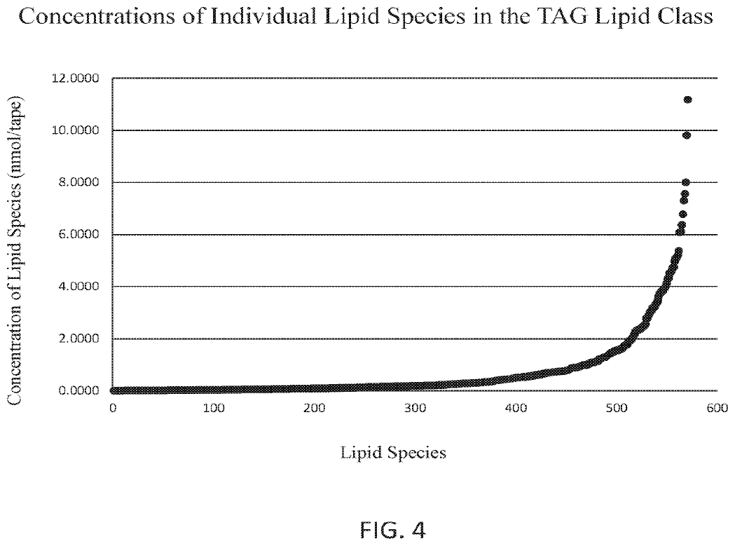

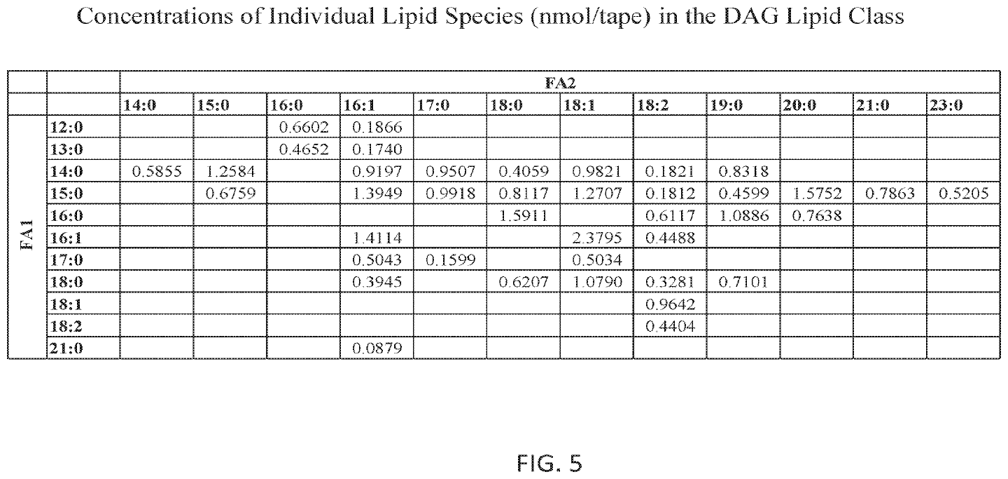

Six sebum tape sebum samples were analyzed using the methods described in Example 1. The amount of 939 lipid species from the lipid classes DAG, TAG, WE, SQ, and FFA was measured, and concentrations were reported in nmol of lipid species per sebum tape (nmol/tape). The concentration of SQ was 201.73185, 62.15865, 36.4587, 227.91105, 137.26815, and 128.3403 nmol/tape, in each of the six sebum samples, respectively. The concentrations of 298 WE lipid species from an exemplary sebum sample are plotted in FIG. 3. The concentrations of 575 TAG lipid species from an exemplary sebum sample are plotted in FIG. 4. The concentrations of 47 DAG lipid species from an exemplary sebum sample are shown in FIG. 5. The concentrations of 18 FFA lipid species from the six sebum samples are shown in Table 5. The lipid class concentrations (reported in nmol/tape) for each of the six samples are reported in Table 6. The lipid class concentration is the sum of the lipid species within the indicated lipid class. The lipid class composition (in %) for each of the six samples is reported in Table 7. The fatty acid concentrations (reported in nmol/tape) for an exemplary sebum sample are shown in Table 8. The fatty acid concentration is the sum of all lipid species within a lipid class containing a specific fatty acid.

TABLE 5
Concentration of FFA Lipid Species.

| | Lipid | Sample | | | | | |
|---|---|---|---|---|---|---|---|
| | Species | 1 | 2 | 3 | 4 | 5 | 6 |
| Lipid | FFA(12:0) | 5.64735 | 0.7347 | 0.79335 | 2.10375 | 7.1925 | 3.2898 |
| Species | FFA(13:0) | 3.6372 | 0.582 | 0.7443 | 2.19585 | 3.5961 | 5.24475 |
| Concentra- | FFA(14:0) | 19.18515 | 4.42065 | 5.96895 | 13.19775 | 18.3555 | 20.14035 |
| tion | FFA(15:0) | 19.4091 | 4.27155 | 7.8096 | 14.1159 | 20.2257 | 20.6403 |
| (nmol/tape) | FFA(16:0) | 25.3818 | 15.42975 | 18.17835 | 21.2484 | 27.2622 | 25.37535 |
| | FFA(16:1) | 17.58285 | 5.0658 | 6.73335 | 13.1091 | 17.8362 | 16.82175 |
| | FFA(17:0) | 13.7064 | 2.778 | 5.31765 | 8.739 | 14.38395 | 15.25845 |
| | FFA(18:0) | 19.26285 | 13.20825 | 15.47535 | 14.9178 | 19.3122 | 19.5684 |
| | FFA(18:1) | 16.9989 | 8.6073 | 11.39355 | 16.8444 | 15.14625 | 14.2683 |
| | FFA(18:2) | 12.1875 | 8.36295 | 8.59965 | 9.47475 | 10.7478 | 10.29855 |
| | FFA(18:3) | 3.6894 | 3.429 | 3.3306 | 3.381 | 3.10875 | 3.1113 |
| | FFA(19:0) | 8.1189 | 1.92375 | 3.09015 | 2.65005 | 4.45365 | 3.7947 |
| | FFA(20:0) | 4.53945 | 1.49955 | 2.6565 | 2.6289 | 4.55835 | 4.317 |
| | FFA(20:4) | 2.4276 | 3.1176 | 3.51525 | 2.0781 | 2.45325 | 2.1507 |
| | FFA(21:0) | 2.23185 | 1.0824 | 1.71105 | 1.2885 | 2.1012 | 2.30265 |
| | FFA(22:0) | 6.1605 | 3.3888 | 4.57305 | 4.02765 | 6.1371 | 5.72505 |
| | FFA(22:6) | 0.4335 | 0.336 | 0.38835 | 0.3306 | 0.40905 | 0.44235 |
| | FFA(23:0) | 3.432 | 0.7764 | 3.23145 | 1.9866 | 4.4991 | 5.5167 |

TABLE 6
Lipid Class Concentrations.

| | Lipid Class Concentration (nmol/tape) | | | | |
|---|---|---|---|---|---|
| Sample | DAG | FFA | SQ | TAG | WE |
| 1 | 52.2495 | 184.0323 | 201.73185 | 132.2994 | 504.8994 |
| 2 | 13.1541 | 79.01445 | 62.15865 | 78.18765 | 389.1858 |
| 3 | 11.34855 | 103.5105 | 36.4587 | 36.73665 | 697.45695 |
| 4 | 22.2669 | 134.3181 | 227.91105 | 331.7958 | 563.6934 |
| 5 | 30.70815 | 181.77885 | 137.26815 | 96.9327 | 798.06435 |
| 6 | 20.7255 | 178.26645 | 128.3403 | 103.78035 | 467.40885 |

TABLE 7
Lipid Class Compositions.

| | Lipid Class Composition (%) | | | | |
|---|---|---|---|---|---|
| Sample | DAG | FFA | SQ | TAG | WE |
| 1 | 4.8595 | 17.1159 | 18.7620 | 12.3045 | 46.9581 |
| 2 | 2.1158 | 12.7094 | 9.9982 | 12.5764 | 62.6002 |
| 3 | 1.2816 | 11.6893 | 4.1172 | 4.1486 | 78.7632 |
| 4 | 1.7396 | 10.4937 | 17.8058 | 25.9218 | 44.0391 |
| 5 | 2.4670 | 14.6036 | 11.0277 | 7.7873 | 64.1143 |
| 6 | 2.3066 | 19.8400 | 14.2835 | 11.5501 | 52.0198 |

TABLE 8
Fatty Acid Concentrations.

Lipid Class

| Fatty acid | WE | DAG | FFA | TAG |
|---|---|---|---|---|
| [FA12:0] | 6.84315 | 0.84675 | 5.64735 | 10.9776 |
| [FA13:0] | 7.2852 | 0.63915 | 3.6372 | 8.68275 |
| [FA14:0] | 32.22165 | 9.40755 | 19.18515 | 49.30515 |
| [FA15:0] | 39.11295 | 14.3124 | 19.4091 | 44.8482 |
| [FA16:0] | 77.70495 | 28.0869 | 25.3818 | 96.95475 |
| [FA16:1] | 154.3581 | 11.7957 | 17.58285 | 78.16755 |
| [FA17:0] | 12.5586 | 5.62185 | 13.7064 | 17.51025 |
| [FA18:0] | 19.44555 | 6.5616 | 19.26285 | 12.5304 |
| [FA18:1] | 27.3681 | 16.8063 | 16.9989 | 56.6478 |
| [FA18:2] | 35.5461 | 3.59685 | 12.1875 | 15.40695 |
| [FA18:3] | 2.47095 | NA | 3.6894 | 0.5235 |
| [FA19:0] | 10.2492 | 3.0903 | 8.1189 | 2.7489 |
| [FA20:0] | 29.3943 | 2.33895 | 4.53945 | 1.41225 |
| [FA20:4] | 0.56625 | NA | 2.4276 | NA |
| [FA21:0] | 9.25695 | 0.8742 | 2.23185 | 0.3852 |
| [FA22:0] | 9.60735 | NA | 6.1605 | 0.711 |
| [FA22:6] | 0.47265 | NA | 0.4335 | NA |
| [FA23:0] | 5.85555 | 0.5205 | 3.432 | 0.08595 |
| [FA24:0] | 12.50865 | NA | NA | NA |
| [FA25:0] | 3.2373 | NA | NA | NA |
| [FA26:0] | 1.25745 | NA | NA | NA |
| [FA27:0] | 1.9755 | NA | NA | NA |
| [FA28:0] | 0.90705 | NA | NA | NA |
| [FA29:0] | 3.97065 | NA | NA | NA |
| [FA30:0] | 0.72525 | NA | NA | NA |

### Example 4: Method Validation (Six Lipid Classes)

In another example, the method was validated for up to six lipid classes in a single injection. The precision of the method for measuring one or more lipid species in one or more lipid classes was evaluated using pooled sebum sample extract. Five technical replicate samples were analyzed. The results are presented in Table 9.

TABLE 9
Precision in sebum (by lipid class).

| | Lipid Class | Lipid Species | Lipid Species |
|---|---|---|---|
| Lipid | Concentrations | Concentrations | Compositions |
| Class | % RSD | Median % RSD | Median % RSD |
| CE | 4.30 | 11.77 | 11.75 |
| TAG | 8.25 | 8.28 | 7.66 |
| DAG | 5.80 | 2.53 | 2.06 |
| FFA | 1.34 | 12.07 | 8.53 |
| WE | 15.07 | 15.07 | N/A |
| SQ | 6.34 | 10.41 | 9.01 |
| All | N/A | 11.37 | 8.52 |

### Example 5: Lipid Analysis (Six Lipid Classes)

Five replicates of pooled sebum sample extract were analyzed using the methods described in Example 1. The amount of 965 lipid species from the lipid classes CE, DAG, TAG, WE, SQ, and FFA was measured, and concentrations were reported in nmol of lipid species per sebum tape (nmol/tape). The concentrations of 26 CE lipid species from the five sebum samples are shown in Table 10. The lipid class concentrations (reported in nmol/tape) for each of the five samples are reported in Table 11. The lipid class concentration is the sum of the lipid species within the indicated lipid class. The lipid class composition (in %) for each of the five samples is reported in Table 12. The fatty acid concentrations (reported in nmol/tape) for an exemplary sebum sample are shown in Table 13. The fatty acid concentration is the sum of all lipid species within a lipid class containing a specific fatty acid.

TABLE 10
Concentration of CE Lipid Species.

| | Lipid | Sample | | | | |
|---|---|---|---|---|---|---|
| | Species | 1 | 2 | 3 | 4 | 5 |
| Lipid | CE(12:0) | 0.0302 | 0.0418 | 0.0456 | 0.0453 | 0.0500 |
| Species | CE(14:0) | 0.2516 | 0.3175 | 0.3332 | 0.3389 | 0.3580 |
| Concentra- | CE(14:1) | 0.2295 | 0.3123 | 0.3137 | 0.3232 | 0.3434 |
| tion | CE(15:0) | 1.0622 | 1.4889 | 1.5407 | 1.5361 | 1.7399 |
| (nmol/tape) | CE(16:0) | 0.9290 | 0.5857 | 0.5249 | 0.4784 | 0.4762 |
| | CE(16:1) | 1.1298 | 1.3312 | 1.3510 | 1.3809 | 1.4726 |
| | CE(17:0) | 0.0923 | 0.1161 | 0.1255 | 0.1271 | 0.1382 |
| | CE(18:0) | 0.1481 | 0.1346 | 0.1238 | 0.1262 | 0.1302 |
| | CE(18:1) | 1.4974 | 0.7615 | 0.6164 | 0.5376 | 0.5122 |
| | CE(18:2) | 2.6752 | 0.9595 | 0.6438 | 0.4839 | 0.3978 |
| | CE(18:3) | 0.1069 | 0.0613 | 0.0522 | 0.0488 | 0.0463 |
| | CE(18:4) | 0.0037 | 0.0029 | 0.0031 | 0.0023 | 0.0027 |
| | CE(20:0) | 0.0695 | 0.0965 | 0.1005 | 0.1076 | 0.1156 |
| | CE(20:l) | 0.0274 | 0.0359 | 0.0359 | 0.0382 | 0.0408 |
| | CE(20:2) | 0.0260 | 0.0319 | 0.0327 | 0.0338 | 0.0336 |
| | CE(20:3) | 0.0694 | 0.0407 | 0.0347 | 0.0304 | 0.0292 |
| | CE(20:4) | 0.3136 | 0.1021 | 0.0621 | 0.0436 | 0.0341 |
| | CE(20:5) | 0.0665 | 0.0294 | 0.0232 | 0.0195 | 0.0190 |
| | CE(22:0) | 0.0117 | 0.0161 | 0.0178 | 0.0178 | 0.0200 |
| | CE(22:1) | 0.0161 | 0.0219 | 0.0225 | 0.0233 | 0.0231 |
| | CE(22:2) | 0.0068 | 0.0089 | 0.0088 | 0.0094 | 0.0100 |
| | CE(22:4) | 0.0074 | 0.0086 | 0.0102 | 0.0088 | 0.0092 |
| | CE(22:5) | 0.0074 | 0.0043 | 0.0050 | 0.0041 | 0.0045 |
| | CE(22:6) | 0.0337 | 0.0134 | 0.0102 | 0.0074 | 0.0067 |
| | CE(24:0) | 0.0136 | 0.0228 | 0.0262 | 0.0261 | 0.0243 |
| | CE(24:1) | 0.0124 | 0.0180 | 0.0211 | 0.0189 | 0.0229 |

TABLE 11
Lipid Class Concentrations.

| | Lipid Class Concentration (nmol/tape) | | | | | |
|---|---|---|---|---|---|---|
| Sample | CE | TAG | DAG | FFA | SQ | WE |
| 1 | 5.88 | 55.13 | 11.93 | 103.39 | 64.91 | 159.74 |
| 2 | 6.17 | 46.38 | 13.06 | 105.71 | 88.46 | 166.29 |
| 3 | 6.14 | 47.75 | 11.93 | 101.97 | 95.34 | 154.12 |
| 4 | 6.57 | 44.99 | 12.36 | 104.33 | 82.93 | 147.86 |
| 5 | 5.97 | 50.82 | 11.14 | 104.51 | 72.64 | 141.41 |

TABLE 12
Lipid Class Compositions.

Lipid Class Composition (%)

| Sample | CE | TAG | DAG | FFA | SQ | WE |
|---|---|---|---|---|---|---|
| 1 | 1.47 | 13.75 | 2.98 | 25.78 | 16.19 | 39.84 |
| 2 | 1.45 | 10.89 | 3.06 | 24.81 | 20.76 | 39.03 |
| 3 | 1.47 | 11.44 | 2.86 | 24.44 | 22.85 | 36.94 |
| 4 | 1.65 | 11.27 | 3.10 | 26.14 | 20.78 | 37.05 |
| 5 | 1.54 | 13.15 | 2.88 | 27.04 | 18.80 | 36.59 |

TABLE 13
Fatty Acid Concentrations.

| | Lipid Class | | | | |
|---|---|---|---|---|---|
| Fatty acid | WE | TAG | CE | DAG | FFA |
| [FA12:0] | 0.99 | 3.03 | 0.04 | 0.12 | 2.15 |
| [FA13:0] | 1.87 | 3.70 | N/A | 0.15 | 2.63 |
| [FA14:0] | 5.25 | 18.91 | 0.32 | 1.84 | 15.65 |
| [FA15:0] | 6.49 | 18.68 | 1.47 | 5.73 | 13.64 |
| [FA16:0] | 11.74 | 40.49 | 0.60 | 6.05 | 37.18 |
| [FA16:1] | 20.89 | 47.11 | 1.33 | 3.28 | 15.74 |
| [FA17:0] | 1.73 | 8.94 | 0.12 | 1.22 | 6.18 |
| [FA18:0] | 1.58 | 4.90 | 0.13 | 2.13 | 30.13 |
| [FA18:1] | 2.84 | 23.45 | 0.79 | 2.48 | 18.23 |
| [FA18:2] | 0.93 | 9.24 | 1.03 | 0.99 | 8.47 |
| [FA18:3] | 0.48 | 0.33 | 0.06 | N/A | 2.06 |
| [FA19:0] | 0.63 | 0.74 | N/A | 0.95 | 5.58 |
| [FA20:0] | 2.02 | 0.66 | 0.10 | 1.66 | 11.17 |
| [FA20:4] | 0.13 | N/A | 0.11 | N/A | 4.53 |
| [FA21:0] | 0.97 | 0.21 | 0.02 | 0.40 | 9.03 |
| [FA22:0] | 1.13 | 0.29 | 0.01 | N/A | 13.12 |
| [FA22:6] | 0.07 | N/A | 0.02 | N/A | 0.94 |
| [FA23:0] | 0.24 | 0.04 | N/A | 0.21 | 4.17 |
| [FA24:0] | 3.83 | N/A | N/A | N/A | N/A |
| [FA25:0] | 0.14 | N/A | N/A | N/A | N/A |
| [FA26:0] | 0.17 | N/A | N/A | N/A | N/A |
| [FA27:0] | 0.10 | N/A | N/A | N/A | N/A |
| [FA28:0] | 0.06 | N/A | N/A | N/A | N/A |
| [FA29:0] | 0.29 | N/A | N/A | N/A | N/A |
| [FA30:0] | 0.07 | N/A | N/A | N/A | N/A |

### Example 6: Sebum Analysis (Six Lipid Classes and TOCs)

In another example, the method was validated for lipid-soluble TOCs and up to six lipid classes in a single injection. The precision of the method for measuring one or more TOCs and/or one or more lipid species in one or more lipid classes was evaluated using pooled sebum sample extract. Three technical replicate samples were analyzed. The precision results for the TOCs and the six lipid classes are presented in Table 14. The precision was also assessed for each tocopherol. Precision was 17.08%, 15.37%, and 14.57% RSD for α-tocopherol, β/γ-tocopherol, and δ-tocopherol, respectively.

TABLE 14
Precision in sebum.

| Lipid | |
|---|---|
| Class/TOC | % RSD |
| CE | 3.99 |
| TAG | 4.95 |
| DAG | 2.85 |
| FFA | 11.99 |
| WE | 1.93 |
| SQ | 5.13 |
| TOC | 15.97 |

Three replicates of pooled sebum sample extract were analyzed using the methods described in Example 1. The amounts of three lipid-soluble TOCs and 965 lipid species from the lipid classes CE, DAG, TAG, WE, SQ, and FFA, were measured, and concentrations were reported in nmol of analyte per sebum tape (nmol/tape). The tocopherols measured in each sample include α-tocopherol, δ-tocopherol, and a combination of β- and γ-tocopherol. The concentrations of tocopherols from the three sebum samples are shown in Table 15. The TOC and lipid class concentrations (reported in nmol/tape) for each of the three replicate samples are reported in Table 16. The lipid class concentration is the sum of the lipid species within the indicated lipid class. The TOC and lipid class composition (in %) for each of the three samples is reported in Table 17. The fatty acid concentration is the sum of all lipid species within a lipid class containing a specific fatty acid.

TABLE 15
Concentration of TOC.

| | TOC Concentration (nmol/tape) | | |
|---|---|---|---|
| Sample | α-tocopherol | β/γ-tocopherol | δ-tocopherol |
| 1 | 0.1023 | 0.0246 | 0.0566 |
| 2 | 0.0993 | 0.0251 | 0.0586 |
| 3 | 0.0738 | 0.0188 | 0.0443 |

TABLE 16
Lipid Class and TOC Concentrations.

| Sam- | Lipid Class and TOC Concentration (nmol/tape) | | | | | | |
|---|---|---|---|---|---|---|---|
| ple | CE | TAG | DAG | FFA | SQ | WE | TOC |
| 1 | 222.26 | 2442.63 | 505.04 | 8630.67 | 101.73 | 160.15 | 0.023 |
| 2 | 239.34 | 2678.12 | 477.09 | 10988.99 | 98.76 | 144.52 | 0.031 |
| 3 | 225.18 | 2485.59 | 489.96 | 9968.92 | 98.14 | 152.02 | 0.03 |

TABLE 17
Lipid Class and TOC Compositions.

| | Lipid Class Composition (%) | | | | | | |
|---|---|---|---|---|---|---|---|
| Sample | CE | TAG | DAG | FFA | SQ | WE | TOC |
| 1 | 1.84 | 20.25 | 4.19 | 71.55 | 0.8434 | 1.33 | 0.0002 |
| 2 | 1.98 | 22.20 | 3.96 | 91.10 | 0.8187 | 1.20 | 0.0003 |
| 3 | 1.87 | 20.61 | 4.06 | 82.64 | 0.8136 | 1.26 | 0.0002 |

1-48. (canceled)

49. A method for determining in a sample, by mass spectrometry, the presence, absence or amount of one or more lipid species from one or more lipid classes, wherein the one or more lipid classes are selected from the group consisting of wax esters (WE), squalene (SQ), triacylglycerols (TAG), diacylglycerols (DAG), free fatty acids (FFA), cholesteryl esters (CE), and combinations thereof, the method comprising:
a) subjecting the sample to an ionization source under conditions suitable to produce one or more ions detectable by mass spectrometry from each of the one or more of the lipid species, wherein the sample is injected into a mass spectrometer without a prior separation or purification step;
b) measuring, by mass spectrometry, the amount of the one or more ions from each of the one or more lipid species; and
c) using the measured amount of the one or more ions to determine the presence, absence or amount of each of the one or more lipid species in the sample.

50. The method of claim 49, wherein the one or more lipid classes comprise wax esters (WE), triacylglycerols (TAG), diacylglycerols (DAG), cholesteryl esters (CE), or combinations thereof, and the method further comprises determining the number of carbons and double bonds of one or more fatty acids of the one or more lipid species.

51. The method of claim 49, wherein the sample is a sebum sample or a sebocyte sample.

52. The method of claim 49, wherein the presence, absence, or amount of two or more lipid species from the one or more lipid classes is determined from a single injection.

53. The method of claim 49, wherein the one or more lipid species from the one or more lipid classes comprise wax esters, and the method further comprises determining the fatty alcohol composition of the wax ester.

54. The method of claim 49, wherein the amount of one or more lipid species from at least two lipid classes is determined.

55. The method of claim 49, wherein one or more internal standards are used to determine the amount of the one or more lipid species in the sample, and wherein the one or more internal standards are selected from one or more lipid classes, which are selected from the group consisting of wax esters (WE), squalene (SQ), triacylglycrides (TAG), diacylglycerols (DAG), free fatty acids (FFA), cholesteryl esters (CE) and combinations thereof.

56. The method of claim 55, wherein the one or more internal standards are selected from the lipid class of wax esters (WE), and the internal standards are selected from the group consisting of WE (FA19:1/OH8:0) and WE (FA17:1/OH8:0).

57. The method of claim 49, wherein the sample is a sebum sample and is collected using sebum tape, swabs, or filter paper.

58. The method of claim 49, further comprising determining the presence, absence or amount of one or more fatty acid isomers in the sample.

59. The method of claim 58, wherein the presence, absence or amount of the one or more fatty acid isomers in the sample is determined using GC-FAME (gas chromatography-fatty acid methyl ester) analysis.

60. The method of claim 49, further comprising determining the presence, absence or amount of one or more tocopherols selected from the group consisting of alpha-tocopherol, beta-tocopherol, gamma tocopherol, and delta-tocopherol.

61. A method for determining in a sample, by mass spectrometry, the presence, absence or amount of:
one or more lipid species from one or more lipid classes, wherein the one or more lipid classes are selected from the group consisting wax esters (WE), squalene (SQ), triacylglycerols (TAG), diacylglycerols (DAG), free fatty acids (FFA), cholesteryl esters (CE), and combinations thereof; and
one or more tocopherols selected from the group consisting of alpha-tocopherol, beta-tocopherol, gamma tocopherol, and delta-tocopherol, and combinations thereof, the method comprising:
a) subjecting the sample to an ionization source under conditions suitable to produce one or more ions detectable by mass spectrometry from each of the one or more of the lipid species and one or more tocopherols, wherein the sample is injected into a mass spectrometer without a prior separation or purification step;
b) measuring, by mass spectrometry, the amount of the one or more ions from each of the one or more lipid species and one or more tocopherols; and
c) using the measured amount of the one or more ions to determine the presence, absence or amount of each of the one or more lipid species and one or more tocopherols in the sample.

62. The method of claim 61, wherein the one or more lipid classes comprise wax esters (WE), triacylglycerols (TAG), diacylglycerols (DAG), cholesteryl esters (CE), or combinations thereof, and the method further comprises determining the number of carbons and double bonds of one or more fatty acids of the one or more lipid species.

63. The method of claim 61, wherein the sample is a sebum sample or a sebocyte sample.

64. The method of claim 61, wherein the presence, absence, or amount of the one or more lipid species from the one or more lipid classes and the presence, absence, or amount of the one or more tocopherols are determined from a single injection.

65. The method of claim 61, wherein the amount of one or more lipid species from at least two lipid classes is determined.

66. A kit comprising one or more internal standards for each of one or more lipid classes selected from the group consisting of wax esters (WE), squalene (SQ), triacylglycrides (TAG), diacylglycerols (DAG), free fatty acids (FFA), cholesterol (CH), cholesteryl esters (CE) and combinations thereof, and packaging material and instructions for using the kit.

67. The kit of claim 66, further comprising one or more internal standards for each of one or more tocopherols (TOC), selected from the group consisting of alpha-tocopherol, beta-tocopherol, gamma tocopherol, and delta-tocopherol, and combinations thereof, and packaging material and instructions for using the kit.
